(19) United States
(12) Patent Application Publication
(10) Pub. No.: US 2021/0315952 A1
(43) Pub. Date: Oct. 14, 2021

(54) Compositions and Methods for Reducing Atmospheric Methane and Nitrous Oxide Emissions

(71) Applicant: Locus IP Company, LLC, Solon, OH (US)

(72) Inventors: Sean FARMER, Ft. Lauderdale, FL (US); Ken ALIBEK, Solon, OH (US); Paul S ZORNER, Encinitas, CA (US)

(21) Appl. No.: 17/267,922

(22) PCT Filed: Oct. 8, 2019
(86) PCT No.: PCT/US2019/055156

§ 371 Date: Feb. 11, 2021

Related U.S. Application Data
(60) Provisional application No. 62/743,167, filed on Oct. 9, 2018.
(60) Provisional application No. 62/885,929, filed on Aug. 13, 2019.

(51) Int. Cl. A61K 36/064; A61K 35/742; A61K 36/07; A61K 38/47; A61K 45/06; A61K 38/12; A61K 9/00; A23K 10/18; A23K 20/189; A23K 20/163; A23K 20/158; A23K 20/147; A23K 10/30; A23K 50/10

(52) U.S. Cl. CPC A61K 36/064; A61K 35/742; A61K 36/07; A61K 38/47; A61K 45/06; A61K 38/12; A23K 50/10; A23K 10/18; A23K 20/189; A23K 20/163; A23K 20/158; A23K 20/147; A23K 10/30; A61K 9/0056

(57) ABSTRACT

The subject invention provides compositions and methods for reducing atmospheric methane and/or nitrous oxide emissions using livestock feed additives and/or supplements. In preferred embodiments, a composition comprising a beneficial microorganism and/or a growth by-product thereof is contacted with animal feed and/or drinking water prior to the animal ingesting it. The composition is capable of, for example, controlling methanogenic microorganisms within the animal's digestive system, and thus, reducing the amount of enteric methane emissions produced from the animal and from the animal's waste.

### CROSS-REFERENCE TO RELATED APPLICATIONS

This applications claims priority to U.S. Provisional Patent App. No. 62/743,167, filed Oct. 9, 2018; and 62/885,929, filed Aug. 13, 2019, each of which is incorporated herein by reference in its entirety.

### BACKGROUND OF THE INVENTION

Gases that trap heat in the atmosphere are called “greenhouse gases,” or “GHG,” and include carbon dioxide, methane, nitrous oxide and fluorinated gases (EPA report 2016 at 6).

Carbon dioxide (CO2) enters the atmosphere through burning fossil fuels (coal, natural gas, and oil), solid waste, trees and wood products, and also as a result of certain chemical reaction, e.g., the manufacture of cement. Carbon dioxide is removed from the atmosphere by, for example, absorption by plants as part of the biological carbon cycle.

Nitrous oxide (N2O) is emitted during industrial activities and during combustion of fossil fuels and solid waste. In agriculture, over-application of nitrogen-containing fertilizers and poor soil management practices can also lead to increased nitrous oxide emissions.

Fluorinated gases including, e.g., hydrofluorocarbons, perfluorocarbons, sulfur hexafluoride, and nitrogen trifluoride are synthetic, powerful greenhouse gases that are emitted from a variety of industrial processes.

Methane (CH4) is emitted during the production and transport of coal, natural gas, and oil. Furthermore, other agricultural practices, and the decay of organic waste in lagoons and municipal solid waste landfills can produce methane emissions. Notably, however, methane emissions also result from production of livestock animals, many of whose digestive systems comprise methanogenic microorganisms (Overview of Greenhouse Gases 2016).

Based on recent measurements from monitoring stations around the world and measurement of older air from air bubbles trapped in layers of ice from Antarctica and Greenland, global atmospheric concentrations of GHGs have risen significantly over the last few hundred years (EPA report 2016 at, e.g., 6, 15).

Especially since the Industrial Revolution began in the 1700s, human activity has contributed to the amount of greenhouse gases in the atmosphere by burning fossil fuels, cutting down forests, and conducting other activities. Many greenhouse gases emitted into the atmosphere remain there for long periods of time ranging from a decade to many millennia. Over time these gases are removed from the atmosphere by chemical reactions or by emissions sinks, such as the oceans and vegetation that absorb greenhouse gases from the atmosphere. Because each greenhouse gas has a different lifetime and a different ability to trap heat in the atmosphere and in order to be able to compare different gases, emissions are generally converted into carbon dioxide equivalents using each gas's global warming potential, which measures how much a given amount of the gas is estimated to contribute to global warming over a period of 100 years after being emitted.

Based on these considerations, the EPA determined that the heating effect caused by greenhouse gases, also termed “radiative forcing,” has increased by about 37% since 1990 (Id. at 16).

Although global emissions of all major greenhouse gases increased between 1990 and 2010, the net emissions of carbon dioxide, which accounts for about three-fourths of the total global emissions, increased by 42%, whereas emissions of methane increased by about 15%, emissions of fluorinated gases doubled, and emission of nitrous oxide emissions increased by about 9% (Id. at 14).

World leaders have attempted to curb the increase of GHG emissions through treaties and other inter-state agreements. One such attempt is through the use of carbon credit systems. A carbon credit is a generic term for a tradable certificate or permit representing the right to emit one ton of carbon dioxide, or an equivalent GHG. In a typical carbon credit system, a governing body sets quotas on the amount of GHG emissions an operator can produce. Exceeding these quotas requires the operator to purchase extra allowances from other operators who have not used all of their carbon credits.

One goal of carbon credit systems is to encourage companies to invest in more green technology, machinery and practices in order to benefit from the trade of these credits. Under the Kyoto Protocol of the United Nations Framework Convention On Climate Change (UNFCCC), a large number of countries have agreed to be bound internationally by policies for GHG reduction, including through trade of emissions credits. While the United States is not bound by the Kyoto Protocol, and while there is no central national emissions trading system in the U.S., some states, such as California and a group of northeastern states, have begun to adopt such trading schemes.

Another attempt to reduce atmospheric GHGs, in particular, methane emissions, has involved the use of feed additives or supplements in livestock production. Ruminant livestock, such as, for example, cattle, sheep, buffalo, goats, deer and camels, have a rumen (or fore-stomach) that contains microorganisms that help digest fibrous plant material. Many of these microorganisms, however, are methanogenic, or methane-producing. As a result of the enteric fermentation of plant matter by these methanogens, the animal releases methane gas into the atmosphere through eructation (Carbon Farming 2018).

By employing methanogen-reducing feed additives and supplements to the feed of livestock, the methanogenic activity within the digestive system can be reduced, thus reducing the emission of methane into the atmosphere. Feed additives to date have included synthetic chemicals, including antibiotics, as well as natural substances, such as tannins, seaweed, fats and oils. Id.

Because global warming may contribute to steeper temperature fluctuations, increased global precipitation, flooding and droughts, as well as changes in sea surface temperature and sea levels, there exists a need to reduce greenhouse gases to slow these detrimental effects.

### BRIEF SUMMARY OF THE INVENTION

The subject invention provides compositions and methods for reducing atmospheric methane and/or nitrous oxide emissions using livestock feed additives and/or supplements. In preferred embodiments, a composition comprising one or more beneficial microorganisms and/or one or more microbial growth by-products is contacted with animal feed and/or drinking water prior to the animal ingesting the feed and/or drinking water. The composition is capable of, for example, controlling methanogenic microorganisms within the animal's digestive system, and thus, reducing the amount of enteric methane emissions produced from the animal and from the animal's waste.

In certain embodiments, the subject invention provides a food composition or a food additive composition, the composition comprising one or more beneficial microorganisms and/or one or more microbial growth by-products. The beneficial microorganisms may be in an active or inactive form. In preferred embodiments, the beneficial microorganisms are non-pathogenic fungi, yeasts and/or bacteria.

In certain embodiments, the composition comprises one or more fungi and/or one or more growth by-products thereof. The fungi can be, for example, Pleurotus spp. fungi, e.g., P. ostreatus (oyster mushrooms), Lentinula spp. fungi, e.g., L. edodes (shiitake mushrooms), and/or Trichoderma spp. fungi, e.g., T. harzianum and/or T. viride. The fungi can be in the form of live or inactive cells, mycelia, spores and/or fruiting bodies. The fruiting bodies, if present, can be, for example, chopped and/or blended into granules and/or a powder form.

In one embodiment, the composition comprises one or more yeasts and/or one or more growth by-products thereof. The yeast(s) can be, for example, Wickerhamomyces anomalus, Saccharomyces spp. (e.g., S. cerevisiae and/or S. boulardii), Starmerella bombicola, Meyerozyma guilliermondii, Pichia occidentalis, Monascus purpureus, and/or Acremonium chrysogenum. The yeast(s) can be in the form of live or inactive cells or spores, as well as in the form of a dried cell mass and/or dormant cells (e.g., a yeast hydrolysate).

In one embodiment, the composition comprises one or more additional beneficial microorganisms, for example, one or more Bacillus spp. bacteria. In certain embodiments, the Bacillus spp. are B. amyloliquefaciens, B. subtilis and/or B. licheniformis.

In an exemplary embodiment, the composition comprises W. anomalus. In an exemplary embodiment, the composition comprises one or more of W. anomalus, P. ostreatus, L. edodes, S. cerevisiae and/or S. boulardii.

In preferred embodiments, the microbe-based composition comprises microbial growth by-products. The composition can comprise the fermentation medium in which the microorganism and/or the growth by-product were produced.

In one embodiment, the growth by-product has been purified from the fermentation medium in which it was produced. Alternatively, in one embodiment, the growth by-product is utilized in crude form. The crude form can take the form of, for example, a liquid supernatant resulting from cultivation of a microbe that produces the growth by-product of interest.

The growth by-products can include metabolites or other biochemicals produced as a result of cell growth, including, for example, amino acids, peptides, proteins, enzymes, biosurfactants, solvents and/or other metabolites.

In one embodiment, the composition comprises lovastatin. Lovastatin is a growth by-product of Pleurotus ostreatus, and inhibits methanogenic archaea via inhibition of the enzyme involved in formation of the isoprenoid building blocks that are essential for methanogen cell membrane synthesis, HMG-CoA reductase. In one embodiment, the composition comprises lovastatin in purified form, either with or without the Pleurotus fungus.

In one embodiment, the composition comprises live Lentinula edodes, which can inhibit HMG-CoA reductase activity without production of lovastatin.

In one embodiment, the composition comprises red yeast rice, or koji, the fermented rice product of Monascus purpureus. Red yeast rice comprises the growth by-product monacolin K, which has a similar structure to lovastatin and has similar ability to inhibit HMG-CoA reductase activity.

In one embodiment, the composition comprises valine. Valine is an amino acid produced by Wickerhamomyces anomalus and Saccharomyces spp., which helps support the growth and health of livestock animals, as well as reduces the amount of nitrogen (e.g., ammonium) excretion by the animals' digestive processes. In one embodiment, the composition comprises valine in purified form, either with or without a yeast that produces it.

In one embodiment, the growth by-product of the subject composition is a biosurfactant, such as, for example, a glycolipid or a lipopeptide. Glycolipids can include, for example, sophorolipids, rhamnolipids, cellobiose lipids, mannosylerythritol lipids and trehalose lipids. Lipopeptides can include, for example, surfactin, iturin, fengycin, arthrofactin and lichenysin.

Advantageously, biosurfactants can have antibacterial properties useful for controlling methanogenic bacteria in ruminant digestive systems.

In one embodiment, the biosurfactant has been purified from the fermentation medium in which it was produced. Alternatively, in one embodiment, the biosurfactant is utilized in crude form. Crude form biosurfactants can take the form of, for example, a liquid mixture comprising biosurfactant sediment in fermentation broth resulting from cultivation of a biosurfactant-producing microbe.

In certain embodiments, the biosurfactant can be added to the composition in the form of a microbial culture containing liquid fermentation broth and cells resulting from submerged cultivation of a biosurfactant-producing microbe. In a specific embodiment, when the biosurfactant is a sophorolipid, this “culture form” biosurfactant can comprise fermentation broth with Starmerella bombicola yeast cells, SLP, and other yeast growth by-products therein. The yeast cells may be active or inactive at the time they are contacted with or formulated with animal food. If a lower concentration of SLP is desired, the SLP portion that results in the S. bombicola culture can be removed, and the residual liquid having, for example, 1-4 g/L residual SLP and, optionally, yeast cells and other growth by-products can be utilized in the subject methods. When use of another biosurfactant is desired, a similar product is envisioned that utilizes any other microbe capable of producing the other biosurfactant.

The microorganisms and/or microbial growth by-products of the subject compositions can be obtained through cultivation processes ranging from small to large scale. These cultivation processes include, but are not limited to, submerged cultivation/fermentation, solid state fermentation (SSF), and modifications, hybrids and/or combinations thereof.

In one embodiment, the subject composition can comprise one or more substances and/or nutrients to supplement animal food and promote health and/or well-being in an animal, such as, for example, sources of amino acids (including essential amino acids), peptides, proteins, vitamins, microelements, fats, fatty acids, lipids, carbohydrates, sterols, enzymes, and trace minerals such as, iron, copper, zinc, manganese, cobalt, iodine, selenium, molybdenum, nickel, fluorine, vanadium, tin and silicon. In some embodiments, the microorganisms of the composition produce and/or provide these substances.

In one embodiment, the composition comprises one or more sources of prebiotics, such as kelp extract, hay, alfalfa, straw, silage, grains and/or legumes.

In certain embodiments, the compositions according to the subject invention can be superior to, for example, purified microbial metabolites alone, due to, for example, the advantageous properties of the yeast cell walls. These properties include high concentrations of mannoprotein and the biopolymer beta-glucan as a part of a yeast cell wall's outer surface. These compounds can serve as, for example, effective emulsifiers. Additionally, the composition can further comprise residual biosurfactants in the culture, as well as other metabolites and/or cellular components, such as solvents, acids, vitamins, minerals, enzymes and proteins. Thus, the compositions can, among many other uses, act as biosurfactants and can have antimicrobial and surface/interfacial tension-reducing properties.

In preferred embodiments, the subject invention provides a method for reducing atmospheric methane and/or nitrous oxide emissions, wherein a composition comprising a beneficial microorganism and/or a growth by-product thereof is contacted with a livestock animal's food and/or drinking water, prior to the animal ingesting the food and/or water. Advantageously, the methods can be useful for, e.g., controlling methanogenic microorganisms inside the animal's digestive system, and thus for reducing the amount of methane produced and/or emitted by the animal and/or its waste. In certain specific embodiments, the livestock animal is a ruminant.

In one embodiment, the composition is ,used either as a liquid or a dried product. In one embodiment the composition is introduced, either in the liquid or dried form, into an animal's food, or into the animal's drinking water as a feed additive and/or supplement.

In one embodiment, the composition is added to standard raw food ingredients utilized in producing processed wet and/or dry animal feed.

In one embodiment, the composition is added to the ingredients utilized in producing, for example, processed dry morsels, kibbles, treats, cakes, nuts, biscuits, or pellets. The supplemented dry food pieces can comprise consistent concentrations of the microbe-based composition per piece. In another embodiment, the composition can be utilized as a surface coating on the dry food pieces. Methods known in the art for producing dry processed food pieces can be used. In certain preferred embodiments, a “cold” pelleting process is used, or a process that does not use high heat or steam.

In one embodiment, the composition is added to dry animal fodder, such as straw, hay, grains or other dry plant-based matter used for feeding livestock animals. In another embodiment, the composition is applied to a pasture as a feed additive for pasture-grazing animals.

In some embodiments, the methods can result in added health benefits for animals, including, for example, enhancing animal growth, enhancing animal immune function, improving absorption of water and of nutrients from food, and improving the health of animals' gut microbiome.

In some embodiments, the methods of the subject invention can be utilized for the reduction in carbon credits used by an operator of a livestock production facility. Thus, in certain embodiments, the subject methods further comprise conducting measurements to assess the effect of the method on the generation of methane emissions and/or to assess the effect of the method on the control of methanogens in the livestock animal's digestive system and/or waste, using standard techniques in the art.

### DETAILED DESCRIPTION OF THE INVENTION

The subject invention provides compositions and methods for reducing atmospheric methane emissions using livestock feed additives and/or supplements. In preferred embodiments, a composition comprising one or more beneficial microorganism and/or one or more microbial growth by-products is contacted with animal feed and/or drinking water prior to the animal ingesting it. The composition is capable of, for example, controlling methanogenic microorganisms within the animal's digestive system, and thus, reducing the amount of enteric methane emissions produced from the animal and from the animal's waste.

Selected Definitions

As used herein, a “biofilm” is a complex aggregate of microorganisms, such as bacteria, wherein the cells adhere to each other and/or to a surface via an extracellular polysaccharide matrix. The cells in biofilms are physiologically distinct from planktonic cells of the same organism, which are single cells that can float or swim in liquid medium.

As used herein, the term “control” used in reference to an undesirable microorganism (e.g., a methanogen) extends to the act of killing, disabling, immobilizing and/or reducing the population numbers of the microorganism, and/or otherwise rendering the microorganism incapable of carrying out the processes that are undesirable (e.g., methane production).

As used herein, a “domesticated” animal is an animal of a species that has been influenced, bred, tamed, and/or controlled over a sustained number of generations by humans, such that a mutualistic relationship exists between the animal and the human. In preferred embodiments, domesticated animals are “livestock,” which include animals raised in an agricultural or industrial setting to produce commodities such as food, fiber and labor. Types of animals included in the term livestock can include, but are not limited to, alpacas, llamas, beef and dairy cattle, bison, pigs, sheep, goats, horses, mules, asses, camels, dogs, chickens, turkeys, ducks, geese, guinea fowl, and squabs.

In certain preferred embodiments, the livestock are “ruminants,” or mammals that utilize a compartmentalized stomach suited for fermenting plant-based foods prior to digestion with the help of a specialized gut microbiome. Ruminants include, for example, bovines (e.g., bison, bongo, buffalo, cow, bull, ox, kudu, imbabala, water buffalo, yak, zebu), sheep, goats, ibex, giraffes, deer, elk, moose, caribou, reindeer, antelope, gazelle, impala, wildebeest, and some kangaroos.

As used herein, “harvested” refers to removing some or all of a microbe-based composition from a growth vessel.

As used herein, an “isolated” or “purified” nucleic acid molecule, polynucleotide, polypeptide, protein, organic compound such as a small molecule (e.g., those described below), or other compound is substantially free of other compounds, such as cellular material, with which it is associated in nature. For example, a purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. A purified or isolated microbial strain is removed from the environment in which it exists in nature. Thus, the isolated strain may exist as, for example, a biologically pure culture, or as spores (or other forms of the strain) in association with a carrier.

In certain embodiments, purified compounds are at least 60% by weight the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

A “metabolite” refers to any substance produced by metabolism (e.g., a growth by-product) or a substance necessary for taking part in a particular metabolic process. A metabolite can be an organic compound that is a starting material, an intermediate in, or an end product of metabolism. Examples of metabolites can include, but are not limited to, enzymes, toxins, acids, solvents, alcohols, proteins, carbohydrates, vitamins, minerals, microelements, amino acids, polymers, and surfactants.

As used herein, a “methanogen” is a microorganism that produces methane gas as a by-product of metabolism. Methanogens are archaea that can be found in the digestive systems and metabolic waste of ruminant animals and non-ruminant animals (e.g., pigs, poultry and horses). Examples of methanogens include, but are not limited to, Methanobacterium spp. (e.g., M. formicicum), Methanobrevibacter spp. (e.g., M. ruminantium), Methanococcus spp. (e.g., M. paripaludis), Methanoculleus spp. (e.g., M. bourgensis), Methanoforens spp. (e.g., M. stordalenmirensis), Methanofollis liminatans, Methanogenium wolfei, Methanomicrobium spp. (e.g., M. mobile), Methanopyrus kandleri, Methanoregula boonei, Methanosaeta spp. (e.g., M. concilii, M. thermophile), Methanosarcina spp. (e.g., M. barkeri, M. mazeii), Methanosphaera stadtmanae, Methanospirillium hungatei, Methanothermobacter spp., and/or Methanothrix sochngenii.

As used herein, reference to a “microbe-based composition” means a composition that comprises components that were produced as the result of the growth of microorganisms or other cell cultures. Thus, the microbe-based composition may comprise the microbes themselves and/or by-products of microbial growth. The microbes may be in a vegetative state, in spore form, in mycelial form, in any other form of microbial propagule, or a mixture of these. The microbes may be planktonic or in a biofilm form, or a mixture of both. The by-products of growth may be, for example, metabolites (e.g., biosurfactants), cell membrane components, expressed proteins, and/or other cellular components. The microbes may be intact or lysed. The cells may be totally absent, or present at, for example, a concentration of at least 1×10⁴, 1×10⁵, 1×10⁶, 1×10⁷, 1×10⁸, 1×10⁹, 1×10¹⁰, 1×10¹¹, 1×10¹², 1×10¹³ or more CFU/ml of the composition.

The subject invention further provides “microbe-based products,” which are products that are to be applied in practice to achieve a desired result. The microbe-based product can be simply the microbe-based composition harvested from the microbe cultivation process. Alternatively, the microbe-based product may comprise further ingredients that have been added. These additional ingredients can include, for example, stabilizers, buffers, carriers (e.g., water or salt solutions), added nutrients to support further microbial growth, non-nutrient growth enhancers and/or agents that facilitate tracking of the microbes and/or the composition in the environment to which it is applied. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and l to 40 in one direction, or 50 to 40, 50 to 30, 50 to 20, and 50 to 10 in the other direction.

As used herein, “reduction” means a negative alteration and “increase” means a positive alteration, wherein the positive or negative alteration is at least 0.25%, 0.5%, 1%, 5%, 10%, 15%, 20%, 25%, 30%, 35%, 40%, 45%, 50%, 55%, 60%, 65%, 70%, 75%, 80%, 85%, 90%, 95% or 100%.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates embodiments “consisting” and “consisting essentially” of the recited component(s).

Unless specifically stated or obvious from context, as used herein, the term “or” is understood to be inclusive. Unless specifically stated or obvious from context, as used herein, the terms “a,” “and” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All references cited herein are hereby incorporated by reference in their entirety.

### Microbe-Based Compositions

In preferred embodiments, the subject invention provides a composition for feeding domesticated animals, the composition comprising one or more beneficial microorganisms and/or one or more microbial growth by-products. The beneficial microorganisms may be in an active or inactive form.

The beneficial microorganisms can be, for example, bacteria, yeasts and/or fungi. These microorganisms may be natural, or genetically modified microorganisms. For example, the microorganisms may be transformed with specific genes to exhibit specific characteristics. The microorganisms may also be mutants of a desired strain. As used herein, “mutant” means a strain, genetic variant or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end.

In one embodiment, the beneficial microorganisms are yeasts and/or fungi. Yeast and fungus species suitable for use according to the current invention, include Acaulospora, Acremonium chrysogenum, Aspergillus, Aureobasidium (e.g., A. pullulans), Blakeslea, Candida (e.g., C. albicans, C. apicola, C. batistae, C. bombicola, C. floricola, C. kuoi, C. riodocensis, C. nodaensis, C. stellate), Cryptococcus, Debaryomyces (e.g., D. hansenii), Entomophthora, Hanseniaspora (e.g., H. uvarum), Hansenula, Issatchenkia, Kluyveromyces (e.g., K. phaffii), Lentinula spp. (e.g., L. edodes), Meyerozyma (e.g., M. guilliermondii), Monascus purpureus, Mortierella, Mucor (e.g., M. piriformis), Penicillium, Phythium, Phycomyces, Pichia (e.g., P. anomala, P. guilliermondii, P. occidentalis, P. kudriavzevii), Pleurotus (e.g., P. ostreatus P. ostreatus, P. sajorcaju, P. cystidiosus, P. cornucopiae, P. pulmonarius, P. tuberregium, P. citrinopileatus and P. flabellatus), Pseudozyma (e.g., P. aphidis), Rhizopus, Rhodotorula (e.g., R. bogoriensis); Saccharomyces (e.g., S. cerevisiae, S. boulardii, S. torula), Starmerella (e.g., S. bombicola), Torulopsis, Thraustochytrium, Trichoderma (e.g., T. reesei, T. harzianum, T. viride), Ustilago (e.g., U. maydis), Wickerhamiella (e.g., W. domericqiae), Wickerhamomyces (e.g., W. anomalus), Williopsis (e.g., W. mrakii), Zygosaccharomyces (e.g., Z. bailii), and others.

In one embodiment, yeast(s) are, for example, Wickerhamomyces anomalus, a Saccharomyces spp. yeast (e.g., S. cerevisiae and/or S. boulardii), Starmerella bombicola, Meyerozyma guilliermondii, Pichia occidentalis, and/or Monascus purpureus. The yeast(s) can be in the form of live or inactive cells or spores, as well as in the form of a dried cell mass and/or dormant cells (e.g., a yeast hydrolysate).

In certain embodiments, the composition comprises live Wickerhamomyces anomalus and/or Saccharomyces spp. yeasts. These yeasts boost acetogenesis and hydrogen utilization by acetogenic bacteria within a ruminant digestive system. Advantageously, this results in less hydrogen availability for methanogenic microorganism to carry out processes in which methane is produced, without negatively affecting the digestive health of the animal. Thus, in one embodiment, the presence of Wickerhamomyces anomalus and/or Saccharomyces spp. yeast. (e.g., S. cerevisiae and/or S. boulardii), and/or growth by-products thereof, in the composition boosts the amount of acetogenic bacteria in a ruminant animal's gut microbiome, and/or decreases the amount of methanogenic bacteria therein.

Additionally, Wickerhamomyces anomalus produces phytase, an enzyme useful for improved digestion and bioavailability of phosphorus from feed, as well as “killer toxins” (e.g., the enzyme exo-β-1,3-glucanase) useful for controlling pathogenic and/or methanogenic microorganisms without causing harm to livestock. This yeast is also able to produce phospholipid biosurfactants.

In some embodiments, the presence of yeast cell biomass further provides a number of proteins (containing up to 50% of dry cell biomass), lipids and carbon sources, as well as a full spectrum of minerals and vitamins (e.g., B1; B2; B3 (PP); B5; B7 (H); B6; E).

In one embodiment, the composition comprises Pleurotus ostreatus, a culture of which can contain concentrations of about 2.5% to 3.0%, or 2.8% lovastatin (dry weight).

Lovastatin is a polyketide growth by-product of Pleurotus, and inhibits methanogenic archaea via inhibition of the enzyme involved in formation of the isoprenoid building blocks that are essential for their cell membrane synthesis, HMG-CoA reductase. Advantageously, lovastatin can inhibit the growth of methanogens without adverse effects on other cellulolytic bacteria in the rumen. In one embodiment, the composition comprises lovastatin in purified form, either with or without the Pleurotus fungus.

In one embodiment, the composition comprises live Lentinula edodes, which can inhibit HMG-CoA reductase activity without production of lovastatin.

In one embodiment, the composition comprises Trichoderma viride and/or Acremonium chrysogenum, which also produce statins similar to lovastatin.

In one embodiment, the composition comprises red yeast rice, or koji, the fermented rice product of Monascus purpureus. Red yeast rice comprises monacolin K, which has a similar structure to lovastatin and has the ability to inhibit HMG-CoA reductase activity.

In one embodiment, the composition comprises synthetic or biologically produced amino acids. In a specific embodiment, the amino acid is valine. Valine is an amino acid produced by Wickerhamomyces anomalus and Saccharomyces spp., which helps support the growth and health of livestock animals, and enables more complete transformation of protein sources in feed to reduce the amount of nitrogen excreted in their waste, in the form of, for example, ammonia. In one embodiment, the composition comprises valine in purified form, either with or without a yeast that produces it.

In certain embodiments, the composition comprises beneficial bacteria, including Gram-positive and Gram-negative bacteria. The bacteria may be, for example Agrobacterium (e.g., A. radiobacter), Azotobacter (A. vinelandii, A. chroococcum), Azospirillum (e.g., A. brasiliensis), Bacillus (e.g., B. amyloliquifaciens, B. firmus, B. laterosporus, B. licheniformis, B. megaterium, Bacillus mucilaginosus, B. subtilis), Frateuria (e.g., F. aurantia), Microbacterium (e.g., M. laevaniformans), Pantoea (e.g., P. agglomerans), Pseudomonas (e.g., P. aeruginosa, P. chlororaphis, P. chlororoaphis subsp. aureofaciens (Kluyver), P. putida), Rhizobium spp., Rhodospirillum (e.g., R. rubrum), and/or Sphingomonas (e.g., S. paucimobilis).

In one embodiment, the composition comprises one or more Bacillus spp. bacteria in the form of spores and/or a dried cell mass. In certain embodiments, the Bacillus spp. are B. amyloliquefaciens, B. subtilis and/or B. licheniformis.

In some embodiments, B. amyloliquefaciens can serve as a probiotic in cattle, to increase body weight gain, increase feed intake and conversion, and increase growth hormone (e.g., GH/IGH-1) levels. Additionally, B. amyloliquefaciens can promote the growth of other beneficial microbes (e.g., producers of short chain fatty acids) while decreasing the amount of potential pathogenic microbes in an animal's gut, e.g., by producing anti-microbial lipopeptide biosurfactants. In some embodiments, a dosage of 4×10¹⁰ CFU/day of B. amyloliquefaciens is administered to an animal as part of a composition of the subject invention.

In some embodiments, B. licheniformis can reduce methane production by methanogens, and inhibit the methanogenic bacteria themselves through production of propionic acid and other metabolites, such as lipopeptide biosurfactants. Additionally, B. licheniformis can help decrease the concentration of ammonia in cattle ruminal fluids while helping increase milk protein production. In pigs, B. licheniformis and B. subtilis can help increase fecal Lactobacillus counts increase the digestibility of nitrogen, and a decrease the emission of ammonia and mercaptans. In some embodiments, a dosage of 2×10¹⁰ CFU/day of B. licheniformis is administered to an animal as part of a composition of the subject invention.

In one embodiment, the microorganism is a strain of B. subtilis, such as, for example, B. subtilis var. locuses B1 or B2, which are effective producers of, for example, surfactin and other lipopeptide biosurfactants. This specification incorporates by reference International Publication No. WO 2017/044953 A1 to the extent it is consistent with the teachings disclosed herein.

In one exemplary embodiment, the composition comprises Wickerhamomyces anomalus, Pleurotus ostreatus, Bacillus amyloliquefaciens and Bacillus licheniformis.

Other microbial strains including strains capable of accumulating significant amounts of, for example, glycolipids, lipopeptides, mannoprotein, beta-glucan, enzymes, and other metabolites that have anti-methanogenic, and/or bio-emulsifying and surface/interfacial tension-reducing properties, can be used in accordance with the subject invention.

In one specific embodiment, the composition comprises about 1×10⁶ to about 1×10¹³, about 1×10⁷ to about 1×10¹², about 1×10⁸ to about 1×10¹¹, or about 1×10⁹ to about 1×10¹⁰ CFU/ml of each species of microorganism present in the composition.

In certain embodiments, the amount of microorganisms in one application of the composition totals about 40 to 70 grams per head (individual animals in a livestock herd), or about 45 to about 65 grams per head, or about 50 to about 60 grams per head.

In one embodiment, the composition comprises about 1 to 100% microorganisms total by volume, about 10 to 90%, or about 20 to 75%.

In one embodiment, the one or more microbial growth by-products of the subject composition is a biosurfactant. Biosurfactants are a structurally diverse group of surface-active substances produced by microorganisms, which are biodegradable and can be efficiently produced using selected organisms on renewable substrates. All biosurfactants are amphiphiles. They consist of two parts: a polar (hydrophilic) moiety and non-polar (hydrophobic) group. Due to their amphiphilic structure, biosurfactants increase the surface area of hydrophobic water-insoluble substances, increase the water bioavailability of such substances, and change the properties of bacterial cell surfaces.

Biosurfactants accumulate at interfaces, thus reducing interfacial tension and leading to the formation of aggregated micellar structures in solution. Safe, effective microbial biosurfactants reduce the surface and interfacial tensions between the molecules of liquids, solids, and gases. The ability of biosurfactants to form pores and destabilize biological membranes permits their use as antibacterial, antifungal, and hemolytic agents. Combined with the characteristics of low toxicity and biodegradability, biosurfactants are advantageous for use in animal feed, additives and supplements.

Biosurfactants include glycolipids, lipopeptides, flavolipids, phospholipids, fatty acid esters, lipoproteins, lipopolysaccharide-protein complexes, and polysaccharide-protein-fatty acid complexes. The common lipophilic moiety of a biosurfactant molecule is the hydrocarbon chain of a fatty acid, whereas the hydrophilic part is formed by ester or alcohol groups of neutral lipids, by the carboxylate group of fatty acids or amino acids (or peptides), organic acid in the case of flavolipids, or, in the case of glycolipids, by the carbohydrate.

Microbial biosurfactants are produced by a variety of microorganisms such as bacteria, fungi, and yeasts in response to the presence of a hydrocarbon source (e.g., oils, sugar, glycerol, etc.) in the growing media. The biosurfactants may be obtained by fermentation processes known in the art.

In one embodiment, the biosurfactant is a glycolipid, a lipopeptide or a phospholipid. Glycolipids can include, for example, sophorolipids, rhamnolipids, cellobiose lipids, mannosylerythritol lipids and trehalose lipids. Lipopeptides can include, for example, surfactin, iturin, fengycin, and lichenysin. Phospholipids can include, for example, cardiolipin.

In certain embodiments, a mixture of biosurfactants is used, comprising a combination of a sophorolipid (including lactonic and/or linear form sophorolipids), a surfactin and/or an iturin (e.g., iturin A).

In certain embodiments, the compositions of the subject invention can comprise the fermentation medium in which the beneficial microorganism and/or the growth by-product was produced. Advantageously, in certain embodiments, this can make the composition superior to, for example, purified microbial metabolites alone, due to, for example, high concentrations of mannoprotein and the biopolymer beta-glucan as a part of a yeast cell wall's outer surface. These compounds can serve as, for example, effective emulsifiers. Additionally, the composition can further comprise residual biosurfactants in the culture, as well as other metabolites and/or cellular components, such as solvents, acids, vitamins, minerals, enzymes and proteins. Thus, the compositions can, among many other uses, act as biosurfactants and can have antimicrobial and surface/interfacial tension-reducing properties.

In one embodiment, the biosurfactant has been purified from the fermentation medium in which it was produced. Alternatively, in one embodiment, the biosurfactant is utilized in crude form. Crude form biosurfactants can take the form of, for example, a liquid mixture comprising biosurfactant sediment in fermentation broth resulting from cultivation of a biosurfactant-producing microbe. This crude form biosurfactant solution can comprise from about 0.001% to 99%, from about 25% to about 75%, from about 30% to about 70%, from about 35% to about 65%, from about 40% to about 60%, from about 45% to about 55%, or about 50% pure biosurfactant.

In certain embodiments, the biosurfactant can be added to the composition in the form of a microbial culture containing liquid fermentation broth and cells resulting from submerged cultivation of a biosurfactant-producing microbe. In a specific embodiment, when the biosurfactant is a sophorolipid, this “culture form” biosurfactant can comprise fermentation broth with Starmerella bombicola yeast cells, sophorolipids (SLP), and other yeast growth by-products therein. The yeast cells may be active or inactive at the time they are contacted with or formulated with animal food. If a lower concentration of SLP is desired, the SLP portion that results in the S. bombicola culture can be removed, and the residual liquid having, for example, 1-4 g/L residual SLP and, optionally, yeast cells and other growth by-products can be utilized in the subject methods. When use of another biosurfactant is desired, a similar product is envisioned that utilizes any other microbe capable of producing the other biosurfactant.

In one embodiment, the composition can further comprise water. For example, the microorganism and/or growth by-products can be mixed with an animal's drinking water as, for example, a feed additive and/or supplement.

In one embodiment, the composition can further comprise pre-made wet or dry animal feed, wherein the pre-made food has been cooked and/or processed to be ready for animal consumption. For example, the microorganism and/or growth by-products can be poured onto and/or mixed with the pre-made food, or the microorganism and/or growth by-products can serve as a coating on the outside of dry animal food pieces, e.g., morsels, kibbles or pellets.

In one embodiment, the composition can further comprise raw ingredients for making animal feed, wherein the raw ingredients, together with the microorganism and/or growth by-products, are then cooked and/or processed to make an enhanced dry or wet feed product.

In certain embodiments, the use of the yeast in the feed provides rich sources of protein and/or polysaccharides. In one embodiment, the subject composition can comprise additional nutrients to supplement an animal's diet and/or promote health and/or well-being in the animal, such as, for example, sources of amino acids (including essential amino acids), peptides, proteins, vitamins, microelements, fats, fatty acids, lipids, carbohydrates, sterols, enzymes, prebiotics, and trace minerals such as, iron, copper, zinc, manganese, cobalt, iodine, selenium, molybdenum, nickel, fluorine, vanadium, tin and silicon.

Preferred compositions comprise vitamins and/or minerals in any combination. Vitamins for use in a composition of this invention can include for example, vitamins A, E, K3, D3, B1, B3, B6, B12, C, biotin, folic acid, panthothenic acid, nicotinic acid, choline chloride, inositol and para-amino-benzoic acid. Minerals can include, for example, salts of calcium, cobalt, copper, iron, magnesium, phosphorus, potassium, selenium and zinc. Other components may include, but are not limited to, antioxidants, beta-glucans, bile salt, cholesterol, enzymes, carotenoids, and many others. Typical vitamins and minerals are those, for example, recommended for daily consumption and in the recommended daily amount (RDA), although precise amounts can vary. The composition would preferably include a complex of the RDA vitamins, minerals and trace minerals as well as those nutrients that have no established RDA, but have a beneficial role in healthy mammal physiology.

### Production of Microorganisms and/or Microbial Growth By-Products

The subject invention utilizes methods for cultivation of microorganisms and production of microbial metabolites and/or other by-products of microbial growth. The subject invention further utilizes cultivation processes that are suitable for cultivation of microorganisms and production of microbial metabolites on a desired scale. These cultivation processes include, but are not limited to, submerged cultivation/fermentation, solid state fermentation (SSF), and modifications, hybrids and/or combinations thereof.

As used herein “fermentation” refers to cultivation or growth of cells under controlled conditions. The growth could be aerobic or anaerobic. In preferred embodiments, the microorganisms are grown using SSF and/or modified versions thereof.

In one embodiment, the subject invention provides materials and methods for the production of biomass (e.g., viable cellular material), extracellular metabolites, residual nutrients and/or intracellular components.

The microbe growth vessel used according to the subject invention can be any fermenter or cultivation reactor for industrial use. In one embodiment, the vessel may have functional controls/sensors or may be connected to functional controls/sensors to measure important factors in the cultivation process, such as pH, oxygen, pressure, temperature, humidity, microbial density and/or metabolite concentration.

In a further embodiment, the vessel may also be able to monitor the growth of microorganisms inside the vessel (e.g., measurement of cell number and growth phases). Alternatively, a daily sample may be taken from the vessel and subjected to enumeration by techniques known in the art, such as dilution plating technique. Dilution plating is a simple technique used to estimate the number of organisms in a sample. The technique can also provide an index by which different environments or treatments can be compared.

In one embodiment, the method includes supplementing the cultivation with a nitrogen source. The nitrogen source can be, for example, potassium nitrate, ammonium nitrate ammonium sulfate, ammonium phosphate, ammonia, urea, and/or ammonium chloride. These nitrogen sources may be used independently or in a combination of two or more.

The method can provide oxygenation to the growing culture. One embodiment utilizes slow motion of air to remove low-oxygen containing air and introduce oxygenated air. In the case of submerged fermentation, the oxygenated air may be ambient air supplemented daily through mechanisms including impellers for mechanical agitation of liquid, and air spargers for supplying bubbles of gas to liquid for dissolution of oxygen into the liquid.

The method can further comprise supplementing the cultivation with a carbon source. The carbon source is typically a carbohydrate, such as glucose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol; fats and oils such as soybean oil, canola oil, rice bran oil, olive oil, corn oil, sesame oil, and/or linseed oil; etc. These carbon sources may be used independently or in a combination of two or more.

In one embodiment, growth factors and trace nutrients for microorganisms are included in the medium. This is particularly preferred when growing microbes that are incapable of producing all of the vitamins they require. Inorganic nutrients, including trace elements such as iron, zinc, copper, manganese, molybdenum and/or cobalt may also be included in the medium. Furthermore, sources of vitamins, essential amino acids, and microelements can be included, for example, in the form of flours or meals, such as corn flour, or in the form of extracts, such as yeast extract, potato extract, beef extract, soybean extract, banana peel extract, and the like, or in purified forms. Amino acids such as, for example, those useful for biosynthesis of proteins, can also be included.

In one embodiment, inorganic salts may also be included. Usable inorganic salts can be potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, lead chloride, copper sulfate, calcium chloride, sodium chloride, calcium carbonate, and/or sodium carbonate. These inorganic salts may be used independently or in a combination of two or more.

In one embodiment, one or more biostimulants may also be included, meaning substances that enhance the rate of growth of a microorganism. Biostimulants may be species-specific or may enhance the rate of growth of a variety of species.

In some embodiments, the method for cultivation may further comprise adding additional acids and/or antimicrobials in the medium before, and/or during the cultivation process. Antimicrobial agents or antibiotics are used for protecting the culture against contamination.

Additionally, antifoaming agents may also be added to prevent the formation and/or accumulation of foam when gas is produced during submerged cultivation.

The pH of the mixture should be suitable for the microorganism of interest. Buffers, and pH regulators, such as carbonates and phosphates, may be used to stabilize pH near a preferred value. When metal ions are present in high concentrations, use of a chelating agent in the medium may be necessary.

The microbes can be grown in planktonic form or as biofilm. In the case of biofilm, the vessel may have within it a substrate upon which the microbes can be grown in a biofilm state. The system may also have, for example, the capacity to apply stimuli (such as shear stress) that encourages and/or improves the biofilm growth characteristics.

In one embodiment, the method for cultivation of microorganisms is carried out at about 5° to about 100° C., preferably, 15 to 60° C., more preferably, 25 to 50° C. In a further embodiment, the cultivation may be carried out continuously at a constant temperature. In another embodiment, the cultivation may be subject to changing temperatures.

In one embodiment, the equipment used in the method and cultivation process is sterile. The cultivation equipment such as the reactor/vessel may be separated from, but connected to, a sterilizing unit, e.g., an autoclave. The cultivation equipment may also have a sterilizing unit that sterilizes in situ before starting the inoculation. Air can be sterilized by methods know in the art. For example, the ambient air can pass through at least one filter before being introduced into the vessel. In other embodiments, the medium may be pasteurized or, optionally, no heat at all added, where the use of low water activity and low pH may be exploited to control undesirable bacterial growth.

In one embodiment, the subject invention further provides a method for producing microbial metabolites such as, for example, biosurfactants, enzymes, proteins, ethanol, lactic acid, beta-glucan, peptides, metabolic intermediates, polyunsaturated fatty acid, and lipids, by cultivating a microbe strain of the subject invention under conditions appropriate for growth and metabolite production; and, optionally, purifying the metabolite. The metabolite content produced by the method can be, for example, at least 20%, 30%, 40%, 50%, 60%, 70%, 80%, or 90%.

The biomass content of the fermentation medium may be, for example, from 5 g/l to 180 g/l or more, or from 10 g/l to 150 g/l. The cell concentration may be, for example, at least 1×10⁹, 1×10¹⁰, 1×10¹¹, 1×10¹²or 1×10¹³ cells per gram of final product.

The microbial growth by-product produced by microorganisms of interest may be retained in the microorganisms or secreted into the growth medium. The medium may contain compounds that stabilize the activity of microbial growth by-product.

The method and equipment for cultivation of microorganisms and production of the microbial by-products can be performed in a batch, a quasi-continuous process, or a continuous process.

In one embodiment, all of the microbial cultivation composition is removed upon the completion of the cultivation (e.g., upon, for example, achieving a desired cell density, or density of a specified metabolite). In this batch procedure, an entirely new batch is initiated upon harvesting of the first batch.

In another embodiment, only a portion of the fermentation product is removed at any one time. In this embodiment, biomass with viable cells, spores, conidia, hyphae and/or mycelia remains in the vessel as an inoculant for a new cultivation batch. The composition that is removed can be a cell-free medium or contain cells, spores, or other reproductive propagules, and/or a combination of thereof. In this manner, a quasi-continuous system is created.

Advantageously, the method does not require complicated equipment or high energy consumption. The microorganisms of interest can be cultivated at small or large scale on site and utilized, even being still-mixed with their media.

### S Preparation of Microbe-based Products

The subject invention provides microbe-based products for reducing the amount of methane emitted as a result of livestock production. One microbe-based product of the subject invention is simply the fermentation medium containing the microorganism and/or the microbial metabolites produced by the microorganism and/or any residual nutrients. The product of fermentation may be used directly without extraction or purification. If desired, extraction and purification can be easily achieved using standard extraction and/or purification methods or techniques described in the literature.

In one embodiment, a yeast fermentation product, designated as “Star 3+,” can be obtained via cultivation of the killer yeast, Wickerhamomyces anomalus, using a modified form of solid state fermentation. The culture can be grown on a substrate with ample surface area onto which the yeasts can attach and propagate, such as, for example, rice, soybeans, chickpeas, pasta, oatmeal or beans. The entire fermentation medium with yeast cells growing throughout, can be harvested after, for example, 3-5 days of cultivation at 25-30° C. The culture can be blended with the substrate, milled and/or micronized, and optionally, dried. This comprises the Star 3+ product. The composition, which can comprise 10¹⁰ to 10¹² cells/gram, can be diluted, for example, 500-1,000 times prior to being mixed with other components.

In an alternative embodiment, the yeast fermentation product is obtained using submerged fermentation, wherein the yeast fermentation product comprises liquid broth comprising cells and any yeast growth by-products. A liquid medium containing necessary sources of carbon, nitrogen, minerals and optionally, antimicrobial substances to prevent contaminating bacterial growth can be used. The culture can be grown with an additional carbon source, particularly, a saturated oil (e.g., 15% canola oil, or used cooking vegetable oil). Typically, the pH begins at 5.0-5.5, then decreases to 3.0-3.5, where it is stabilized. The fermentation broth with cells and yeast growth by-products, which can be harvested after, for example, 24-72 hours of cultivation at 25-30° C., comprises this alternative form of the Star 3+ product.

In one embodiment, a yeast fermentation product can be obtained via submerged cultivation of the biosurfactant-producing yeast, Starmerella bombicola. This yeast is an effective producer of glycolipid biosurfactants, such as SLP. The fermentation broth after 5 days of cultivation at 25° C. can contain the yeast cell suspension and, for example, 150 g/L or more of SLP. This yeast fermentation product can be further modified if less biosurfactant is desired in the cleaning composition. For example, fermentation of S. bombicola results in precipitation of the SLP into a distinguishable layer. This SLP layer can be removed and the residual liquid and biomass, which can still contain 1-4 g/L of residual SLP, can then be utilized in the subject cleaning composition.

The microorganisms in the microbe-based product may be in an active or inactive form. Furthermore, the microorganisms may be removed from the composition, and the residual culture utilized. The microbe-based products may be used without further stabilization, preservation, and storage. Advantageously, direct usage of these microbe-based products preserves a high viability of the microorganisms, reduces the possibility of contamination from foreign agents and undesirable microorganisms, and maintains the activity of the by-products of microbial growth.

The microbes and/or medium (e.g., broth or solid substrate) resulting from the microbial growth can be removed from the growth vessel and transferred via, for example, piping for immediate use.

In one embodiment, the microbe-based product is simply the growth by-products of the microorganism. For example, biosurfactants produced by a microorganism can be collected from a submerged fermentation vessel in crude form, comprising, for example about 50% pure biosurfactant in liquid broth.

In other embodiments, the microbe-based product (microbes, medium, or microbes and medium) can be placed in containers of appropriate size, taking into consideration, for example, the intended use, the contemplated method of application, the size of the fermentation vessel, and any mode of transportation from microbe growth facility to the location of use. Thus, the containers into which the microbe-based composition is placed may be, for example, from 1 gallon to 1,000 gallons or more. In other embodiments the containers are 2 gallons, 5 gallons, 25 gallons, or larger.

Upon harvesting, for example, the yeast fermentation product, from the growth vessels, further components can be added as the harvested product is placed into containers and/or piped (or otherwise transported for use). The additives can be, for example, buffers, carriers, other microbe-based compositions produced at the same or different facility, viscosity modifiers, preservatives, nutrients for microbe growth, tracking agents, solvents, biocides, other microbes and other ingredients specific for an intended use.

Other suitable additives, which may be contained in the formulations according to the invention, include substances that are customarily used for such preparations. Examples of such additives include surfactants, emulsifying agents, lubricants, buffering agents, solubility controlling agents, pH adjusting agents, preservatives, stabilizers and ultra-violet light resistant agents.

In one embodiment, the product may further comprise buffering agents including organic and amino acids or their salts. Suitable buffers include citrate, gluconate, tartarate, malate, acetate, lactate, oxalate, aspartate, malonate, glucoheptonate, pyruvate, galactarate, glucarate, tartronate, glutamate, glycine, lysine, glutamine, methionine, cysteine, arginine and a mixture thereof. Phosphoric and phosphorous acids or their salts may also be used. Synthetic buffers are suitable to be used but it is preferable to use natural buffers such as organic and amino acids or their salts listed above.

In a further embodiment, pH adjusting agents include potassium hydroxide, ammonium hydroxide, potassium carbonate or bicarbonate, hydrochloric acid, nitric acid, sulfuric acid or a mixture.

In one embodiment, additional components such as an aqueous preparation of a salt such as sodium bicarbonate or carbonate, sodium sulfate, sodium phosphate, sodium biphosphate, can be included in the formulation.

Advantageously, in accordance with the subject invention, the microbe-based product may comprise broth in which the microbes were grown. The product may be, for example, at least, by weight, 1%, 5%, 10%, 25%, 50%, 75%, or 100% broth. The amount of biomass in the product, by weight, may be, for example, anywhere from 0% to 100% inclusive of all percentages therebetween.

Optionally, the product can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C. On the other hand, a biosurfactant composition can typically be stored at ambient temperatures.

### Local Production of Microbe-Based Products

In certain embodiments of the subject invention, a microbe growth facility produces fresh, high-density microorganisms and/or microbial growth by-products of interest on a desired scale. The microbe growth facility may be located at or near the site of application. The facility produces high-density microbe-based compositions in batch, quasi-continuous, or continuous cultivation.

The microbe growth facilities of the subject invention can be located at the location where the microbe-based product will be used (e.g., a free-range cattle pasture). For example, the microbe growth facility may be less than 300, 250, 200, 150, 100, 75, 50, 25, 15, 10, 5, 3, or 1 mile from the location of use.

Because the microbe-based product can be generated locally, without resort to the microorganism stabilization, preservation, storage and transportation processes of conventional microbial production, a much higher density of microorganisms can be generated, thereby requiring a smaller volume of the microbe-based product for use in the on-site application or which allows much higher density microbial applications where necessary to achieve the desired efficacy. This allows for a scaled-down bioreactor (e.g., smaller fermentation vessel, smaller supplies of starter material, nutrients and pH control agents), which makes the system efficient and can eliminate the need to stabilize cells or separate them from their culture medium. Local generation of the microbe-based product also facilitates the inclusion of the growth medium in the product. The medium can contain agents produced during the fermentation that are particularly well-suited for local use.

Locally-produced high density, robust cultures of microbes are more effective in the field than those that have remained in the supply chain for some time. The microbe-based products of the subject invention are particularly advantageous compared to traditional products wherein cells have been separated from metabolites and nutrients present in the fermentation growth media. Reduced transportation times allow for the production and delivery of fresh batches of microbes and/or their metabolites at the time and volume as required by local demand.

The microbe growth facilities of the subject invention produce fresh, microbe-based compositions, comprising the microbes themselves, microbial metabolites, and/or other components of the medium in which the microbes are grown. If desired, the compositions can have a high density of vegetative cells or propagules, or a mixture of vegetative cells and propagules.

In one embodiment, the microbe growth facility is located on, or near, a site where the microbe-based products will be used (e.g., a livestock production facility), preferably within 300 miles, more preferably within 200 miles, even more preferably within 100 miles. Advantageously, this allows for the compositions to be tailored for use at a specified location. The formula and potency of microbe-based compositions can be customized for specific local conditions at the time of application, such as, for example, which animal species is being treated; what season, climate and/or time of year it is when a composition is being applied; and what mode and/or rate of application is being utilized.

Advantageously, distributed microbe growth facilities provide a solution to the current problem of relying on far-flung industrial-sized producers whose product quality suffers due to upstream processing delays, supply chain bottlenecks, improper storage, and other contingencies that inhibit the timely delivery and application of, for example, a viable, high cell-count product and the associated medium and metabolites in which the cells are originally grown.

Furthermore, by producing a composition locally, the formulation and potency can be adjusted in real time to a specific location and the conditions present at the time of application. This provides advantages over compositions that are pre-made in a central location and have, for example, set ratios and formulations that may not be optimal for a given location.

The microbe growth facilities provide manufacturing versatility by their ability to tailor the microbe-based products to improve synergies with destination geographies. Advantageously, in preferred embodiments, the systems of the subject invention harness the power of naturally-occurring local microorganisms and their metabolic by-products to improve GHG management.

The cultivation time for the individual vessels may be, for example, from 1 to 7 days or longer. The cultivation product can be harvested in any of a number of different ways.

Local production and delivery within, for example, 24 hours of fermentation results in pure, high cell density compositions and substantially lower shipping costs. Given the prospects for rapid advancement in the development of more effective and powerful microbial inoculants, consumers will benefit greatly from this ability to rapidly deliver microbe-based products.

### Methods for Reducing Atmospheric Methane Emissions

In preferred embodiments, the subject invention provides a method for reducing atmospheric methane and/or nitrous oxide emissions, wherein a composition comprising one or more beneficial microorganisms and/or one or more microbial growth by-products is contacted with a livestock animal's food and/or drinking water, prior to the animal ingesting the food and/or water. Advantageously, in certain embodiments, the methods can control methanogenic microbes in the animal's digestive system, as well as in the animal's waste, while reducing the need for antibiotics.

As used herein, “reduction” refers to a negative alteration, and the term “increase” refers to a positive alteration, wherein the negative or positive alteration is at least 0.25%, 0.5%, 1%, 5%, 10%, 15%, 20%, 25%, 30%, 35%, 40%, 45%, 50%, 55%, 60%, 70%, 75%, 80%, 85%, 90%, 95%, or 100%.

In some embodiments, the desired reduction is achieved within a relatively short time period, for example, within 1 week, 2 weeks, 3 weeks or 4 weeks. In some embodiments, the desired reduction is achieved within, for example, 1 month, 2 months, 3 months, 4 months, 5 months or 6 months after employing the subject methods. In some embodiments, the desired reduction is achieved within 1 year, 2 years, 3 years, 4 years, or 5 years after employing the subject methods.

In some embodiments, prior to contacting a composition with food or water, the method comprises assessing a livestock animal or livestock production facility for local conditions, determining a preferred formulation for the composition (e.g., the type, combination and/or ratios of microorganisms and/or growth by-products) that is customized for the local conditions, and producing the composition with said preferred formulation.

The local conditions can include, for example, age, health, size and species of the animal; purpose for producing the animal (e.g., meat, fur, fiber, eggs, labor, milk, etc.); species within the microbial population of an animal's gut; environmental conditions, such as amount and type of GHG emissions at a facility, current climate, and/or season/time of year; mode and/or rate of application of the composition, and others as are deemed relevant.

After assessment, a preferred formulation for the composition can be determined so that the composition can be customized for these local conditions. The composition is then cultivated, preferably at a microbe growth facility that is within 300 miles, preferably within 200 miles, even more preferably within 100 miles of the location of application (e.g., the animal or livestock production facility).

In some embodiments the local conditions are assessed periodically, for example, once annually, biannually, or even monthly. In this way, the composition formula can be modified in real time as necessary to meet the needs of the changing local conditions.

In one embodiment, the composition is used according to the subject methods as either a liquid or a dried product. In one embodiment the composition is introduced, either in the liquid or dried form, into an animal's food, or into the animal's drinking water.

In one embodiment, the composition is added to standard raw food ingredients utilized in wet and/or dry animal feed.

As used herein, “dry food” refers to food that contains a limited moisture content, typically in the range of about 5% to about 15% or 20% w/v. Typically, dry processed food comes in the form of small to medium sized individual pieces, e.g., morsels, kibbles, treats, biscuits, nuts, cakes or pellets.

In one embodiment, the composition can be added to the raw ingredients utilized in producing dry food, such as grains, vegetables, fruits, dried plant matter, and other flavorings, additives and/or sources of nutrients. The supplemented dry food pieces can comprise consistent concentrations of the microbe-based composition per piece. In another embodiment, the composition can be utilized as a surface coating on the dry food pieces. Methods known in the art for producing dry processed foods can be used, including pressurized milling, extrusion, and/or pelleting.

In an exemplary embodiment, dry food may be prepared by, e.g., screw extrusion, which includes cooking, shaping and cutting raw ingredients into a specific shape and size in a very short period of time. The ingredients may be mixed into homogenous expandable dough and cooked in an extruder, and forced through a die under pressure and high heat. After cooking, the pellets are then allowed to cool, before optionally being sprayed with a coating. This coating may comprise, for example, liquid fat or digest, including liquid or powdered hydrolyzed forms of an animal tissue such as liver or intestine from, e.g., chicken or rabbit, and/or a nutritional oil. In other embodiments, the pellet is coated using a vacuum enrobing technique, wherein the pellet is subjected to vacuum and then exposed to coating materials after which the release of the vacuum drives the coating materials inside the pellet. Hot air drying can then be employed to reduce the total moisture content to 10% or less.

In one embodiment, the dry food is produced using a “cold” pelleting process, or a process that does not use high heat or steam. The process can use, for example, liquid binders with viscous and cohesive properties to hold the ingredients together without risk of denaturing or degrading important components and/or nutrients in the compositions of the subject invention.

In one embodiment, the composition can be applied to animal fodder, or cut and dried plant matter, such as hay, straw, silage, sprouted grains, legumes and/or grains.

In one embodiment, where the livestock animal is a pasture grazing animal, the composition can be introduced onto the pasture ground cover (e.g., grass, clover, legumes, forbs) upon which the animal is feeding. The composition can be sprayed, sprinkled, poured, or applied over a broad surface of pasture land using standard agricultural and/or landscaping techniques, for example, through irrigation and/or fertilization systems.

In one embodiment, the composition may be prepared as a spray-dried biomass product. The biomass may be separated by known methods, such as centrifugation, filtration, separation, decanting, a combination of separation and decanting, ultrafiltration or microfiltration.

In one embodiment, the composition has a high nutritional content, for example, comprising up to 50% protein, as well as polysaccharides, vitamins, and minerals. As a result, the composition may be used as part of all of a complete animal feed composition. In one embodiment, the feed composition comprises the subject composition ranging from 15% of the feed to 100% of the feed.

The subject compositions may be rich in at least one or more of fats, fatty acids, lipids such as phospholipid, vitamins, essential amino acids, peptides, proteins, carbohydrates, sterols, enzymes, and trace minerals such as, iron, copper, zinc, manganese, cobalt, iodine, selenium, molybdenum, nickel, fluorine, vanadium, tin and silicon.

In some embodiments, the compositions described herein can be co-administered with another feed composition as a dietary supplement. The dietary supplement can have any suitable form such as a gravy, drinking water, beverage, yogurt, powder, granule, paste, suspension, chew, morsel, liquid solution, treat, snack, pellet, pill, capsule, tablet, sachet, or any other suitable delivery form. The dietary supplement can comprise the subject microbe-based compositions, as well as optional compounds such as vitamins, minerals, probiotics, prebiotics, and antioxidants. In some embodiments, the dietary supplement may be admixed with a feed composition or with water or other diluent prior to administration to the animal.

In certain embodiments, the animal feed composition can comprise nutrients for promoting the health of an animal.

In one embodiment, the nutrient is a fat and/or an amino acid. In one embodiment, the nutrient is a protein. In one embodiment, the nutrient is a vitamin or a trace mineral. Vitamins can include, for example, vitamins A, E, K3, D3, B1, B3, B6, B12, C, biotin, folic acid, panthothenic acid, nicotinic acid, choline chloride, inositol and para-amino-benzoic acid. Minerals can include, for example, salts of calcium, cobalt, copper, iron, magnesium, phosphorus, potassium, selenium and zinc. Other components may include, but are not limited to, antioxidants, beta-glucans, bile salt, cholesterol, enzymes, carotenoids, and many others.

According to the methods of the subject invention, administration of the microbe-based compositions can be performed as part of a dietary regimen, which can span a period ranging from parturition through the adult life of the animal. In certain embodiments, the animal is a young or growing animal. In some embodiments, the animal is an aging animal. In other embodiments administration begins, for example, on a regular or extended regular basis, when the animal has reached more than about 30%, 40%, 50% , 60%, or 80% of its projected or anticipated lifespan.

The compositions described herein are administered to an animal via the animal's food and/or drinking water, for a time required to accomplish one or more objectives of the invention, such as, a reduction in the amount of methane emissions produced from the animal, without being a detriment to the quality of life, health and wellness of the animal. In some embodiments, the compositions described herein are contacted with an animal's food and/or drinking water on a regular basis, e.g., at every meal, or one meal per day.

In some embodiments, the methods can result in added health benefits for animals, including, for example, enhancing animal growth, enhancing animal immune function, improving absorption of water and of nutrients from food, and improving the health of animals' gut microbiome by increasing the percentage of beneficial gut microorganisms and/or decreasing the percentage of detrimental gut microorganisms in the animal's digestive system.

In some embodiments, the methods of the subject invention can be utilized for the reduction in carbon credits used by an operator of a livestock production facility. Thus, in certain embodiments, the subject methods further comprise conducting measurements to assess the effect of the methods on the generation of methane emissions and/or the reduction in methanogenic microorganisms in the animal's digestive system. These measurements can be conducted according to known methods in the art (see, e.g., Storm et al. 2012, incorporated herein by reference), including, for example, gas capture and quantification, chromatography, respiration chambers (which measure the amount of methane exhaled by an individual animal), and in vitro gas production technique (where feed is fermented under controlled laboratory and microbial conditions to determine amount of methane emitted per gram of dry matter). The measurements can also come in the form of testing the microbial population in an animal, for example, by sampling milk, feces, and/or stomach contents and using, for example, DNA sequencing and/or cell plating to determine the number of methanogenic microbes present therein.

Measurements can be conducted at a certain time point after application of the microbe-based composition. In some embodiments, the measurements are conducted after about 1 week or less, 2 weeks or less, 3 weeks or less, 4 weeks or less, 30 days or less, 60 days or less, 90 days or less, 120 days or less, 180 days or less, and/or 1 year or less.

Furthermore, the measurements can be repeated over time. In some embodiments, the measurements are repeated daily, weekly, monthly, bi-monthly, semi-monthly, semi-annually, and/or annually.

### EXAMPLES

A greater understanding of the present invention and of its many advantages may be had from the following examples, given by way of illustration. The following examples are illustrative of some of the methods, applications, embodiments and variants of the present invention. They are not to be considered as limiting the invention. Numerous changes and modifications can be made with respect to the invention.

### Example 1—Production of Sophorolipids

The fermentation temperature is generally between about 22-28° C., depending on the microorganism and/or microbial growth by-product being cultivated. For Starmerella bombicola, a temperature of about 25° C. is optimum.

The pH should be from about 2.0 to about 7.0, and preferably between about 3.0 to about 6.5, depending on the microorganism and/or microbial growth by-product being cultivated. Additionally, in order to reduce the possibility of contamination, the cultivation process can begin at a first pH range and then be adjusted to a second pH range either higher or lower than the first pH range.

Under these cultivation conditions, industrially useful production of biomass, biosurfactants and other metabolites are achieved after as little as 24 hours of fermentation. Upon completion of the fermentation, the growth by-products and/or leftover culture can then be harvested from the reactors and applied for a variety of industrial purposes.

The reactors can then be sterilized again and re-used for fermenting either the same microbe-based products or different microbe-based products. For example, the reactors can be used to cultivate Starmerella bombicola for production of SLPs, sterilized, and then used to produce SLPs again, or to cultivate, for example, Pseudozyma aphidis for production of, for example, MELs.

The subject systems can be used to produce sophorolipids (SLPs) on an industrial scale and without contamination of the production culture.

In one embodiment, the reactor is inoculated with Starmerella bombicola yeast. The culture medium comprises a carbon source, a lipid, a nitrogen source, and can be supplemented with up to 200 ppm pure sophorolipid.

The yeast and culture medium are incubated at pH 3.0-3.5 under aerobic conditions and for a period of time sufficient for initial accumulation of biomass (typically about 24 hours to about 48 hours). The temperature is held at 22° to 28° C. and dissolved oxygen concentration is held within 15% to 30% (of 100% ambient air). Once initial biomass accumulation is achieved, pH is adjusted to 5.5 and the process continued.

When the culture acidifies to pH 3.5, the fermentation process continues, keeping the pH stable at this level until sufficient accumulation of SLP is achieved in the medium. The SLP can form, for example, a brown-colored translucent to opaque sediment layer in the medium. The SLP is then recovered from the fermentation medium, and the leftover yeast fermentation product can be harvested separately.

### REFERENCES

Government of Western Australia. (2018). “Carbon farming: reducing methane emissions from cattle using feed additives.” https://www.agric.wa.gov.au/climate-change/carbon-farming-reducing-methane-emissions-cattle-using-feed-additives. (“Carbon Farming 2018”).

Pidwirny, M. (2006). “The Carbon Cycle”. Fundamentals of Physical Geography, 2nd Edition. Viewed Oct. 1, 2018. http://www.physicalgeography.net/fundamentals/9r.html. (“Pidwirny 2006”).

Storm, Ida M.L.D., A.L.F. Hellwing, N. I. Nielsen, and J. Madsen. (2012). “Methods for Measuring and Estimating Methane Emission from Ruminants.” Animals (Basel). June 2(2): 160-183. doi: 10.3390/ani2020160.

United States Environmental Protection Agency. (2016). “Climate Change Indicators in the United States.” https://www.epa.gov/sites/production/files/2016-08/documents/climate_indicators_2016.pdf. (“EPA Report 2016”).

United States Environmental Protection Agency. (2016). “Overview of Greenhouse Gases.” Greenhouse Gas Emissions. https://www.epa.gov/ghgemissions/overview-greenhouse-gases. (“Greenhouse Gas Emissions 2016”).

1. A method for reducing atmospheric methane emissions, wherein a composition comprising a beneficial microorganism and/or a microbial growth by-product, is contacted with a livestock animal's food and/or drinking water prior to the animal ingesting the food and/or drinking water,
wherein the microorganism is Wickerhamomyces anomalus, Bacillus licheniformis, Bacillus amyloliquefaciens, Bacillus subtilis, Starmerella bombicola, Pichia occidentalis, Pleurotus ostreatus, Lentinula edodes, Monascus purpureus, Trichoderma harzianum, Trichoderma viride, Acremonium chrysogenum, Saccharomyces cerevisiae, and/or Saccharomyces boulardii,
wherein the livestock animal is provided with the food and/or drinking water and ingests said food and/or water, and
wherein the livestock animal's ingestion of the composition allows the composition to contact a methanogenic microorganism present in the livestock animal's digestive system and control said methanogenic microorganism.

2. The method of claim 1, wherein the composition comprises fermentation broth in which the microorganism was cultivated.

3. The method of claim 1, comprising one or more microorganisms selected from Wickerhamomyces anomalus, Bacillus licheniformis, Bacillus amyloliquefaciens, and Pleurotus ostreatus.

4. The method of claim 1, wherein the growth by-product is an enzyme capable of controlling methanogenic microorganisms.

5. The method of claim 4, wherein the enzyme is exo-β-1,3-glucanase.

6. The method of claim 1, wherein the growth by-product is an HMG-CoA inhibitor.

7-8. (canceled)

9. The method of claim 1, wherein the growth by-product is a biosurfactant.

10. The method of claim 9, wherein the biosurfactant is a glycolipid selected from sophorolipids, rhamnolipids, cellobiose lipids, mannosylerythritol lipids, and trehalose lipids.

11. The method of claim 9, wherein the biosurfactant is a lipopeptide selected from surfactin, iturin, fengycin, arthrofactin and lichenysin.

12-15. (canceled)

16. The method of claim 1, wherein the composition is mixed with raw ingredients used for producing processed dry animal feed, and wherein the raw ingredients are processed and/or cooked to form morsels, pellets, kibbles, cakes, nuts, treats or biscuits.

17. The method of claim 1, wherein the composition is mixed with animal fodder selected from dry hay, straw, grains, legumes and silage.

18-20. (canceled)

21. The method of claim 1, further comprising conducting measurements to assess the effect of the method on the generation of methane emissions and/or to assess the effect of the method on the control of methanogens in the animal's digestive system and/or waste.

22. (canceled)

23. A composition for feeding a domesticated animal, the composition comprising a microorganism and/or a microbial growth by-product, wherein the microorganism is Wickerhamomyces anomalus, Bacillus licheniformis, Bacillus amyloliquefaciens, Bacillus subtilis, Starmerella bombicola, Pichia occidentalis, Pleurotus ostreatus, Lentinula edodes, Monascus purpureus, Trichoderma harzianum, Trichoderma viride, Acremonium chrysogenum, Saccharomyces cerevisiae, and/or Saccharomyces boulardii.

24. The composition of claim 23, wherein the growth by-product is a glycolipid biosurfactant selected from sophorolipids, rhamnolipids, cellobiose lipids, mannosylerythritol lipids, and trehalose lipids.

25. The composition of claim 23, wherein the growth by-product is a lipopeptide selected from surfactin, iturin, fengycin, arthrofactin and lichenysin.

26. The composition of claim 23, wherein the growth by-product is in crude form, said crude form comprising the growth by-product in fermentation broth in which it was produced.

27. (canceled)

28. The composition of claim 23, comprising one or more microorganisms selected from Wickerhamomyces anomalus, Bacillus licheniformis, Bacillus amyloliquefaciens and Pleurotus ostreatus.

29. The composition of claim 23, wherein the growth by-product is an enzyme capable of controlling methanogenic microorganisms.

30. The composition of claim 29, wherein the enzyme is exo-β-1,3-glucanase.

31. The composition of claim 23, wherein the growth by-product is an HMG-CoA inhibitor.

32-33. (canceled)

34. The composition of claim 23, further comprising one or more nutrients selected from sources of amino acids, petides, proteins, vitamins, microelements, fats, fatty acids, lipids, carbohydrates, sterols, enzymes, and trace minerals.

35. (canceled)
